(19) United States
(12) Patent Application Publication
(10) Pub. No.: US 2014/0037557 A1
(43) Pub. Date: Feb. 6, 2014

(54) Dental Whitening Method

(71) Applicant: Dermaceutical Products, Inc., Hauppauge, NY (US)

(72) Inventors: Jennifer Y. Wang, Dix Hills, NY (US); Charles Huzak, Woodbury, NY (US)

(73) Assignee: Dermaceutical Products, Inc., Hauppauge, NY (US)

(21) Appl. No.: 13/957,400

(22) Filed: Aug. 1, 2013

Related U.S. Application Data
(60) Provisional application No. 61/678,196, filed on Aug. 1, 2012.

(51) Int. Cl. A61K 8/23; A61Q 11/00; A61K 8/42; A61K 8/22; A61K 8/19

(52) U.S. Cl. CPC A61K 8/23; A61K 8/22; A61K 8/19; A61K 8/42; A61Q 11/00
USPC 424/53; 424/49

(57) ABSTRACT

A two-step system for whitening stained or discolored teeth by applying separately, and sequentially, to the teeth (i) a bleaching activator followed by (ii) a bleaching agent. In preferred embodiments the bleaching activator is a transition metal catalyst and the bleaching agent is a peroxide.

### CONTINUING APPLICATION DATA

The present application claims priority from U.S. Provisional Patent Application Ser. No. 61/678,196, filed Aug. 1, 2012, the contents of which are hereby incorporated by reference in their entirety.

### STATEMENT REGARDING FEDERALLY SPONSORED RESEARCH OR DEVELOPMENT

Not applicable.

### FIELD OF INVENTION

The present invention relates to a method for whitening teeth, especially teeth that are stained and/or discolored.

### BACKGROUND OF THE INVENTION

Tooth discoloration may be caused by a number of factors, including age-related tooth changes, extrinsic staining (e.g., from diet or smoking), intrinsic staining (e.g., associated with use of tetracycline), and color change secondary to tooth trauma

Peroxides are well-known oxidizing agents. When exposed to peroxide at a sufficient concentration and for a sufficient duration, organic dyes and pigments lose their ability to absorb light and, therefore, appear white.

In oral care, peroxides—hydrogen peroxide and carbamide peroxide—are placed in contact with teeth to oxidize both internal and external organic stains, rendering the teeth whiter.

Means for applying peroxides to the teeth for purposes of whitening are known in the art and include dentrifice and similar compositions in gel and paste forms (the latter known in the art as “prophy paste”). Strips and adhesive films can be impregnated with peroxide. Additionally, peroxide-containing gels can be held in contact with teeth in custom-fabricated bleaching trays. Non-limiting, illustrative examples of the above applications means are described in U.S. Pat. No. 6,824,704 (tooth whitening gels containing high percentage of hydrogen peroxide) and U.S. Pat. No. 6,884,426 (tooth whitening film containing high concentration of peroxide). (To the extent pertinent, the disclosures of all granted US patents and published US patent applications cited are incorporated in their entirety by reference.)

There are, however, physicochemical limitations to effective use of peroxides in oral care. For example, as disclosed in the U.S. Pat. No. 4,728,455, bleaching efficacy of peroxide drops sharply below 60° C., which is well above body temperature. In order to overcome this limitation, high concentrations of peroxide are often applied in a manner that allows extended contact time with the teeth. However, the high concentrations used in extracoronal tooth bleaching are known to cause short-term tooth sensitivity and/or gingival irritation. See, American Dental Association, Council on Scientific Affairs, “Tooth Whitening/Bleaching: Treatment Considerations for Dentists and Their Patients”, September 2009.

In professional settings, attempts are made to mitigate these negative sequelae, including by protecting the sensitive soft tissues with a ligated rubber dam, and, in many instances, applying heat to accelerate or intensify the oxidizing process. (As noted above, the efficacy of peroxide in bleaching is impeded at temperatures below 60° C.). Use of heat lamps is also subject to limitations in terms of patient discomfort. High levels of heat cannot be endured for prolonged periods. Accordingly, teeth bleaching treatments with high concentrations of peroxide in combination with heat typically require multiple visits to the dentist's office (e.g., 4 to 10 appointments) before significant changes can be seen.

Other prior art teeth bleaching techniques utilize light, sometimes in combination with heat, to accelerate the bleaching process. For example, U.S. Pat. No. 5,032,178 discloses the use of a 30-35% aqueous solution of hydrogen peroxide with a dental curing light. U.S. Pat. No. 7,802,988 discloses a method that utilizes laser light to activate bleaching agents applied to the teeth. Light-enhanced approaches to teeth whitening suffer from limitations similar to heat-based treatment modalities. Both are not only expensive, but also are limited to clinical settings, in part because of the need to position and focus the heat or light source in a manner that will minimize irritation and damage to soft tissue of the gum.

In laundry and hard surfacing cleansing applications, bleach catalysts or heavy metal bleach activators have been employed to achieve satisfactory bleaching at lower peroxide concentration and lower wash temperatures. U.S. Pat. Nos. 4,728,455, 5,114,611, and 6,022,490 disclose laundry compositions containing a catalyst for the bleaching action of the peroxide compound comprising a water-soluble complex of manganese (III) with a multi-dentate ligand. U.S. Patent Nos. 6,119,705, 5,804,542, 5,798,326, 5,705,464 and 5,703,030 disclose dishwashing compositions comprising cobalt III catalysts.

Adoption of catalyst-based approaches from laundry and hard surface cleaning to personal care has been very limited. In large part this is due to differences in formulation requirements and conditions of use. Laundry and hard cleansing applications are typically conducted at a pH of about 11. Oral care applications require a much lower pH.

Moreover, while powders are widely used in laundry and hard surface cleaning, in oral care, peroxide must be incorporated into a liquid, gel or paste composition.

Examples of catalyst-based teeth whitening applications include the following.

U.S. Pat. No. 6,290,935 discloses a two-component whitening dentifrice composition that is described as achieving rapid whitening of stained or discolored teeth.

The dentifrice composition is comprised of a first component containing a peroxide compound and the second component containing Fe ion implanted silicate clay. The first and second components are maintained separately from each other until dispensed.

U.S. Pat. No. 6,488,913 discloses a tooth bleaching system based on the combination of hydrogen peroxide (in a free form or in the form of its adduct with urea) and peroxide and/or percarbonates of metals belonging to the first or second group of the periodic table. Mixing hydrogen peroxide, carbamide peroxide, or hydrogen peroxide/urea adduct with a metal peroxide and/or percarbonate is described as producing “accelerated generation of radical oxygen”, and thereby a “faster teeth bleaching action”.

U.S. Pat. No. 5,648,064 discloses a two-component whitening dentifrice composition containing, as a first component, a peroxide compound and, as a second component, a manganese coordination complex compound such as manganese gluconate, which activates the peroxide compound and accelerates the release of active oxygen to accelerate whitening action. The two components, while intended to be stored separately prior to dispense, are taught to be co-dispensed and admixed to form a single composition.

The patent and clinical literature recognizes many drawbacks, limitations and deficiencies of prior art methods of peroxide-based methods of whitening stained or discolored tech. Application of peroxide at high concentrations can cause gum irritation, teeth sensitization, and even weaken or damage the enamel. Moreover, many prior art peroxide-based oral care whitening methods can only be administered in a clinical setting, often in combination with heat to provide an optimal temperature for the oxidizing activity of peroxide. Accordingly, there has been and remains a need for a method of whitening teeth that that can be accomplished at a lower concentration of peroxide, within a shorter period of time and/or with fewer treatments, at body temperature, which can be administered at home. These needs are met by the two-step method of the present invention.

### SUMMARY OF THE INVENTION

The present invention is directed to a two-step system for whitening teeth by applying separately, and sequentially, to the teeth (i) a bleaching activator followed by (ii) a bleaching agent or (i) a bleaching agent followed by (ii) a bleaching activator. Applicants have surprisingly and unexpectedly discovered that separate, sequential application of a bleaching activator, followed by a bleaching agent, especially a peroxide at low concentrations, achieved superior whitening in comparison to applying a bleaching activator and a bleaching agent in the same composition.

In a preferred embodiment, the bleaching activator is applied first, and nearly instantaneously, within one minute, a bleaching agent is applied.

### BRIEF DESCRIPTION OF THE DRAWINGS

FIG. 1 is a series of photographs showing bleaching efficacies with a 1.25% hydrogen peroxide solution under different conditions. Similarly prepared samples of a tea stained strip were treated as follows. Sample A was dipped into water with a pH of 10. Sample B was dipped into a Bleaching Agent Solution (1.25% hydrogen peroxide). Sample C was dipped into a Bleaching Activator Solution (2.5 ppm manganese sulfate and 0.1% sodium gluconate), followed by being dipped into the Bleaching Agent Solution. Sample D was dipped into the Bleaching Agent Solution, followed by being dipped in the Bleaching Activator Solution. Sample E was dipped into a premixed solution of equal parts Bleaching Agent Solution and Bleaching Activator Solution.

FIG. 2 is a series of photographs showing the bleaching efficacies of a 2.5% sodium percarbonate solution under different conditions. Similarly prepared samples of a tea stained strip were treated as follows. Sample A was dipped into water with a pH of 10. Sample B was dipped into a Bleaching Agent Solution (2.5% sodium percarbonate). Sample C was dipped into a Bleaching Activator Solution (2.5 ppm manganese sulfate and 0.1% sodium gluconate), followed by being dipped into the Bleaching Agent Solution. Sample D was dipped into the Bleaching Agent Solution, followed by being dipped in the Bleaching Activator Solution. Sample E was dipped into a premixed solution of equal parts Bleaching Agent Solution and Bleaching Activator Solution.

FIG. 3 is a schematic from the VITA SYSTEM 3D-MASTER®, a method of assessing tooth lightening.

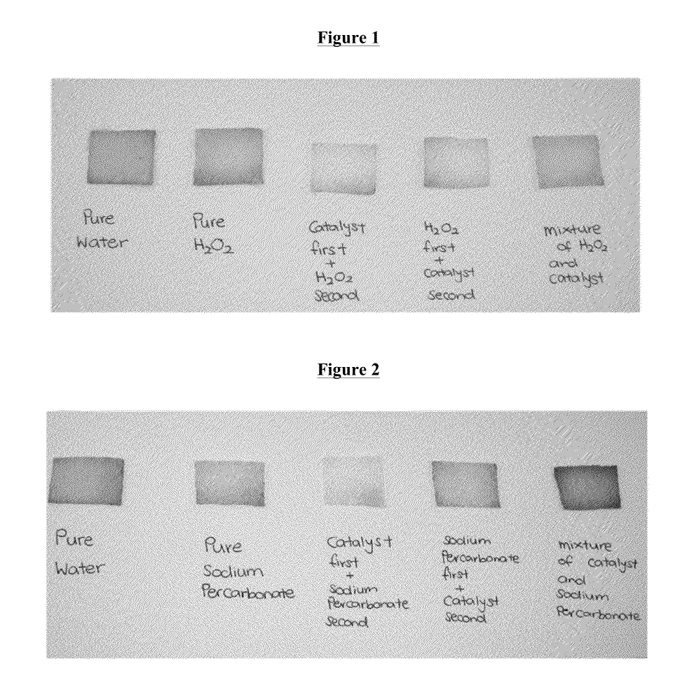

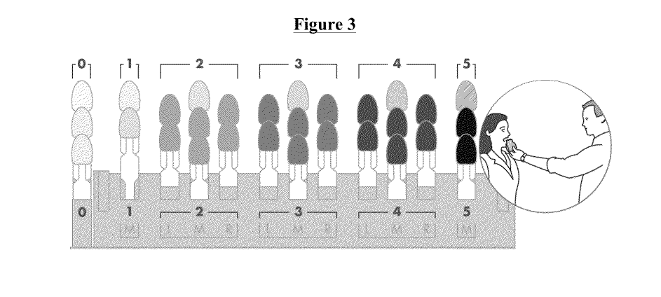

### DETAILED DESCRIPTION OF THE INVENTION

The present invention is directed to a method for whitening stained or discolored teeth comprising the separate and sequential steps of applying to the teeth (i) a bleaching activator and (ii) a bleaching agent.

In a first embodiment, the present invention is directed to a method of whitening stained or discolored teeth having as a first step applying a bleaching activator to the teeth, followed by a second step of applying a bleaching agent to the teeth that have been treated with a bleaching activator in the first step.

In a second embodiment, the present invention is directed to a method of whitening stained or discolored teeth having as a first step applying a bleaching agent to the teeth, followed by a second step of applying a bleaching activator to the teeth that have been treated with a bleaching agent in the first step.

As used herein, by the phrase “whitening” is meant reducing the amount of staining or discoloration of teeth, or lightening the shade of teeth. While many methods of measuring lightness and shade are available, including the use of digital chromameters, a preferred and widely accepted methodology for assessing tooth lightening in a standardized manner is the VITA SYSTEM 3D-MASTER® available from VITA Zahnfabrik H. Rauter GmbH & Co. KG.

The VITA SYSTEM 3D-MASTER® system is described in U.S. Pat. No. 5,498,157, the disclosure of which is incorporated herein by reference in its entirety. Lightness level (value) is determined by holding a shade guide to the mouth of the subject and selecting/matching the color of the subject's teeth with one of five groupings, designated 0, 1, 2, 3, 4, or 5. Differences within a lightness level reflect the various degrees of chroma and hue.

As used herein and unless otherwise expressly noted or required by the context, all percentages refer to percentages by weight (wt-%).

As used herein in connection with a measured quantity, for example weight, “about” refers to that variation in the measured quantity as would be expected by one skilled in the art exercising a level of care commensurate with the objective of the measurement and the equipment used, and includes uncertainties that may be introduced by mathematical rounding errors.

Bleaching agents suitable for use in the methods of the present invention include materials capable of releasing an oxidizing agent such as free radical oxygen ions. Bleaching agents include peroxides, metal chlorites and bromides, perborates, percarbonates, peroxyacids, and combinations thereof.

Preferred bleaching agents are peroxides, including but not limited to hydrogen peroxide, carbamide peroxide, sodium perborate, and sodium percarbonate.

As will be appreciated by the skilled artisan, the concentration of peroxide will vary depending on a number of factors including the duration, frequency and technique of application, and the identity of the intended user. For example, the concentration of peroxide will differ if a composition is designed for professional use only, for application by the user/patient but under professional control, or if it is to be broadly available to the public for in-home, non-supervised use. Peroxides may be used in the methods of the present invention at peroxide concentrations ranging from about 0.05% to about 50%, preferably from about 0.1% to about 30%, and still more preferably from about 0.2% to about 15%, where peroxide concentration is understood to be H₂O₂%. In every 100 ml of a 3% solution, 3% (3 ml) will be pure hydrogen peroxide and 97 ml will be water. Due to its potential for irritation of the gingiva, peroxides are preferably used at concentrations of less than about 15% by weight.

Bleaching activators suitable for use in the methods of the present invention are (i) transition metal complexes, wherein the transition metal is selected from the group consisting of manganese, cobalt, silver, tin and iron, and (ii) bases.

By “transition metal complex” is meant a transition metal coordinated (bonded to) one or more ligands (neutral or anionic non-metal species).

Preferably, the bleaching activator is used in the methods of the present invention at a concentration ranging from 1 ppm to about 3% by weight, preferably from 5ppm to 5,000 ppm, and more preferably from about 50 ppm to about 1,000 ppm.

Non-limiting examples of transition metal complexes suitable for use in the methods of the present invention are disclosed in the following granted patents: U.S. Pat. No. 4,728,455; U.S. Pat. No. 5,114, 611; U.S. Pat. No. 6,022,490; U.S. Pat. No. 6,119,705; and U.S. Pat. No. 5,804,542.

In aspects of the present invention where the bleaching activator is a base, the base preferably can raise the pH of composition containing the bleaching agent, preferably peroxide, to a pH of 10 and above.

The methods of the present invention may be practiced using any of the modalities known in the art for applying oral care compositions. As discussed above, the present invention is directed to separate, sequential application of two oral care compositions, the first composition containing a bleaching activator, the second composition containing a bleaching agent. These compositions, which may be in the form of a prophy paste, dentrifice, gel, or whitening strip, may be delivered or applied to the surface of teeth via a toothbrush, a swab, a finger cot, tube applicator (e.g., doe foot), bleaching tray, or dental instrument.

In addition to the bleaching agent or bleaching activator, the oral care compositions used in practicing the methods of the present invention will normally include water, humectant, thickener, surfactant and polishing agent, non-limiting examples of which include Hydrated Silica, Glycerin, Sorbitol, PVM/MA Copolymer, Sodium Lauryl Sulfate, Cellulose Gum, Sodium Hydroxide, Propylene Glycol, Carrageenan, Sodium Saccharin, Titanium Dioxide, Acrylates Copolymer, Disodium EDTA, Poloxamer 407, PVP, Pullulan, Shellac, Mono and Diglycerides, Silica, PEG 75, Polysorbate 20, Potassium Phosphate, Disodium Phosphate, Caprylic/Capric Triglyceride, Locust Bean Gum, Triacetin, Xanthan Gum, Xylitol, FD&C Dyes, Diatomaceous Earth.

In one aspect of the present invention, the bleaching agent and the bleaching activator may be applied simultaneously (i.e., in a single application) provided that the bleaching activator is encapsulated, for example, in a plurality of shear sensitive beads.

The encapsulated bleaching activator can be released upon application of a shear force, such as toothbrush or ultrasonic device. Non-limiting examples of pressure-rupturable encapsulation suitable for use in the compositions and methods of the present invention are described in U.S. Pat. No. 3,936,573. The outermost layer of the encapsulating material can be any of the many known organic, hydrophobic, polymeric, film-forming materials including polyacrylic, polyurethane, polyethylene, polyamide, synthetic or nature wax and combinations thereof

Alternatively, the bleaching activator or the bleaching agent may be encapsulated in a polymeric delivery system or a liposomal delivery system.

In one preferred embodiment, the polymeric delivery system is a porous, non-swellable, rigid, beads or microspheres that are chemically and biologically inert. The particles are loaded with peroxide that is held inside the pores by capillary forces. The pores are interconnected and open to the particle surface such that peroxide in the internal pore space can be released to the exterior of the particle via diffusion. Polymeric delivery system of this type are available from AMCOL Health and Beauty Solutions under the commercial names, Microsponge®, Poly-Pore® and Poly-Trap®. Poly-Pore® is further described in U.S. Pat. Nos. 5,830,960, 5,834,577 and 6,248,849. Poly-Trap® ® is further described in U.S. Patent Nos. 4,962,133 and 4,962,170.

Microsponge® particles are composed of a cross-linked copolymer of monoethylenically unsaturated monomers and polyethylenically unsaturated monomers. These particles have a cross-linking density from 20% to 80%, are substantially spherical in shape, and have an average diameter of about 1 micron to about 100 microns, a total pore volume of about 0.01 cc/g to about 4.0 cc/g, a surface area of about 1 m²/g to about 500 m²/g, and an average pore diameter of about 0.001 micron to about 3.0 microns. U.S. Pat. Nos. 4,690,825 and 4,873,091 further describe the Microsponge® PMDS. U.S. Pat. Nos. 4,690,825 and 5,145,675 describe processes for loading an active ingredient, here a peroxide, into the Microsponge®.

In another embodiment of this aspect of the invention, the peroxide is encapsulated in a liposome. Liposomes are microscopic spherical vesicles formed by hydrating phospholipids, including lecithins, phosphatidyl ethanol amines, or sphingomyelins. By varying their lipid content, size, surface charge and method of preparation, liposomes can be designed to deliver specific active ingredients, including retinol, at desired rates of release.

In the compositions used in the teething whitening methods of the present invention, liposomes based on lecithins are preferred. Lecithins are phospholipids, more particularly, fatty acid diesters of the choline ester of glycerophosphoric acid. Fatty acid diesters of the choline ester of glycerophosphoric acid are commonly referred to as phosphatidyl cholines. Soy lecithin is a preferred fatty acid diester of the choline ester of glycerophosphoric acid.

Liposomes useful in the practice of certain embodiments of the present invention contain about 5-15 mole per cent of negatively charged phospholipids, such as phospatidyl-glycerol, phospatidylserine or phospatidylinositol. Liposomes can be sized (e.g., particle size range and particle size distribution) by methods known in the art.

Methods of preparing liposomes are well known in the art and typically involve low-sheer mixing of phospholipids in water. In the resulting liposomal dispersions, phospholipids arrange themselves in multi-lamellar sheets of concentric phospholipid spheres in a hydrophilic head-to-head and hydrophobic tail-to-tail configuration. See, e.g., M. Rosen, ed., Delivery System Handbook for Personal Care and Cosmetic Products: Technology, Applications and Formulations, pp. 285-303 (William Andrew, 2005).

The following formulation examples are illustrative of the methods of the present invention for whitening stained or discolored teeth. The components and specific ingredients of the example formulations are presented as being typical, and various modifications can be derived in view of the foregoing disclosure within the scope of the invention.

### EXAMPLE 1

### Tea Bag Stained Swatches

A staining solution is prepared by using three bags of Lipton Black Tea boiled in 100 grams of deionized water for 15 minutes. After the solution has cooled down, water is added (to replace evaporate) and yield a final staining solution of 100 grams.

Tea stained strips are prepared as the follows. A white absorbent substrate is cut into 2×2 squares (swatches). A non-limiting example of a suitable white absorbent substrate is sold under the trade name of Creatology™. The squares are submerged and soaked in the staining solution for ten minutes. After removal, the squares are allowed to air dry.

Two Bleaching Agent Solutions are prepared: the first, 1.25% by weight hydrogen peroxide; the second, 2.5% by weight sodium percarbonate.

A Bleaching Activator solution containing 12.5 ppm manganese sulfate and 0.1% sodium gluconate is also prepared.

As a control, a solution of water, adjusted to a pH of 10, is also prepared.

The tea stained test squares are photographed to establish baseline color (degree of staining) and are then dipped (submerged) into one or more of the above solutions for ten minutes. The square is then removed and photographed. Color change (lightening/whitening) is determined using a standardized visual grading scale, e.g., the Gardner scale.

Sample A is dipped into water with a pH of 10. Sample B is dipped into a Bleaching Agent Solution (1.25% hydrogen peroxide). Sample C is dipped into a Bleaching Activator Solution (2.5 ppm manganese sulfate and 0.1% sodium gluconate), followed by being dipped into the Bleaching Agent Solution. Sample D is dipped into the Bleaching Agent Solution, followed by being dipped in the Bleaching Activator Solution. Sample E is dipped into a premixed solution of equal parts Bleaching Agent Solution and Bleaching Activator Solution.

The above experiment is repeated with a different Bleaching Agent Solution, (2.5% sodium percarbonate). The same Bleaching Activator Solution is used.

In both examples, no color change was observed in Samples A and B had no color change indicating 1.25% hydrogen peroxide and 2.5% sodium percarbonate alone at room temperature has no bleaching power. However, it was surprisingly discovered that the premixed solution (Sample E) had no bleaching power, whereas samples C and D had effective bleaching power at the very low concentrations of peroxides.

### EXAMPLE 2

### Whitening Strips

Begin with a strip of polyethylene film, preferably having a thickness of about 0.013 mm, an array of shallow pockets, the pockets measuring about 0.4 mm across and about 0.1 min deep. The strip has a flexural stiffness of about 0.6 grams/cm as measured on a Handle-O-Meter, model #211-300, available from Thwing-Albert Instrument Co. of Philadelphia, Pa., as per test method ASTM D2923-95. The above-described strip readily conforms to the teeth by either lightly pressing the strip against the teeth and/or by applying the strip to the teeth and then gently sucking through the gaps between the teeth. The wearer easily removes the strip by peeling it off. The strips may be left in contact with the teeth for a period ranging from about 5 minutes to about 120 minutes per day, preferably from about 30 minutes to about 60 minutes.

Bleaching Agent Composition. In a first vessel combine the following ingredients: (i) 70% PEG-8; (ii) 5% hydroxypropylcellulose, (iii) 10% carbamide peroxide. Next, add 15% water. Mix until homogenous.

Bleaching Activator Composition. In a second vessel combine the following ingredients: (i) 79.89875% PEG-8 and (ii) 5% hydroxypropylcellulose to create a gel. Next, add a solution of (i) 0.1% Sodium Gluconate, (ii) 0.00125% Manganese Sulfate, and (iii) 15% water.

Four groups of strips are prepared. A first group of strips is left uncoated, without either a Bleaching Agent Composition or a Bleaching Activator Composition. A second group of mono-layer strips is coated with the Bleaching Agent Composition. A third group of mono-layer strips is coated with the Bleaching Activator Composition. A fourth group of bi-layer strips is coated with both the Bleaching Agent Composition and the Bleaching Activator Composition.

Fifty patients having stained/discolored teeth are recruited to participate in a study to assess the tooth whitening efficacy of the methods of the present invention. The fifty patients are divided into five groups, respectively Groups A, B, C, D and E.

Each patient in Group A is provided with two sets of 28 strips. The first set of strips is coated only with the Bleaching Agent Composition. The second set of strips is coated only with a Bleaching Activator Composition.

Each patient in Group B is provided with two sets of 28 strips. The first set of strips is coated only with the Bleaching Agent Composition. The second set of strips, however, is not coated with a Bleaching Activator Composition.

Each patient in Group C is provided with two sets of 28 strips. The first set of strips is not coated with the Bleaching Agent Composition. The second set of strips is coated only with a Bleaching Activator Composition.

Each patient in Group D is provided with two sets of 28 strips. The first set of strips is coated with both the Bleaching Agent Composition and the Bleaching Activator Composition. The second set of strips is uncoated.

Each patient in Group E is provided with two sets of 28 strips. The first set of strips is uncoated. The second set of strips is also uncoated.

Prior to beginning the study, a baseline measurement of tooth whiteness of each patient is taken using the VITA SYSTEM 3D-MASTER®. Each patient applies and leaves the first strip on his/her teeth for about one minute. After one minute, the patient removes (peels off) the first strip and then applies the second strip to his/her teeth. The second strip is left on his/her teeth for about 30 minutes and is then removed. The above procedure is repeated once daily for 28 consecutive days. After 28 days, a second measurement of tooth whiteness is made using the same VITA SYSTEM 3D-MASTER®. Surprisingly, participants in Group A (Bleaching Agent Composition followed by Bleaching Activator Composition) show a surprisingly greater improvement in tooth whiteness in comparison to Group D (combination of Bleaching Agent Composition and Bleaching Activator Composition).

### EXAMPLE 3

### Prophy Paste

Combination Bleaching Agent/Bleaching Activator Prophy Paste Treatment.

To a suitable mixing vessel add, in order, (i) 42.9% PEG-8; (ii) 3.5% sodium silicate; (iii) 1% sodium fluoride; (iv) 47.25% pumice; (v) 0.25% silica; (vi) 5% sodium percarbonate; (vii) 0.1% sodium gluconate, and (viii) 0.00125% manganese sulfate. Mix until homogenous.

Two-Part Prophy Paste Treatment.

Part 1—Bleaching Activator Paste. To a suitable mixing vessel add, in order, (i) 10% sodium gluconate; (ii) 0.12% manganese sulfate, and (iii) 54.88% water. Mix until homogenous. Then add 35% SD alcohol. Part 2.

Part 2—Bleaching Agent Paste. To a suitable mixing vessel add, in order, (i) 46.75% PEG-8; (ii) 3% sodium silicate; (iii) 1% sodium fluoride; (iv) 44% pumice, (v) 0.25% silica, and (vi) 5% sodium percarbonate. Mix until homogenous.

Thirty patients having stained/discolored teeth are recruited to participate in a study to assess the tooth-whitening efficacy of the methods of the present invention. The thirty patients are divided into three groups, respectively Groups A, B, and C. Patients in Group A are treated with the Combination Bleaching Agent/Bleaching Activator Prophy Paste. Patients in Group B are treated first with Part 1—Bleaching Activator Paste and then with Part 2—Bleaching Agent Paste. Patients in Group C are treated first with Part 2—Bleaching Agent Paste then Part 1—Bleaching Activator Paste.

Prior to receiving treatment, a baseline assessment of tooth whiteness of each patient is taken using the VITA SYSTEM 3D-MASTER®. The paste compositions as described above are applied to each patient with a polisher and prophy angle similar to PerfectPearl™ disposable prophy angle by Sybron Dental Specialities, Inc. After treatment, a second assessment of tooth whiteness of each patient is taken using the VITA SYSTEM 3D-MASTER®.

Surprisingly and unexpectedly, participants in Group B (Bleaching Activator followed by Bleaching Agent) and Group C (Bleaching Agent then Bleaching Activator) show a surprisingly greater improvement in tooth whiteness in comparison to Group A (Combination Bleaching Agent/Bleaching Activator Prophy Paste).

It is to be understood that the method of the invention disclosed is a preferred embodiment thereof and that various changes and modifications may be made therein without departing from the spirit of the invention or scope as defined in the following claims.

what is claimed is:

1. A two-step method for whitening stained or discolored teeth comprising the sequential and separate steps of first applying a bleaching activator in a first oral care composition and then applying a bleaching agent in a second oral care composition.

2. The method of claim 1 wherein the bleaching agent is selected from the group of peroxides, metal chlorites and bromides, perborates, percarbonates, peroxyacids, and combinations thereof.

3. The method of claim 2 wherein the bleaching agent is a peroxide.

4. The method of claim 3 wherein the peroxide is selected from the group consisting of hydrogen peroxide, carbamide peroxide, sodium perborate, and sodium percarbonate.

5. The method of claim 4 wherein the peroxide is present in an oral care composition at a peroxide concentration ranging from about 0.2% to about 15%.

6. The method of claim 5 wherein the bleaching activator is a transition metal complex and the transition metal is selected from the group consisting of manganese, cobalt, silver, tin and iron.

7. The method of claim 6 wherein the transition metal complex is manganese sulfate.

8. The method of claim 6 wherein the transition metal complex is present in an oral care composition at a concentration on weight basis of from about 5 ppm to about 5,000 ppm.

9. The method of claim 1 wherein the first oral care composition and the second oral care composition are in the form of a paste, dentrifice, or gel.

10. The method of claim 9 wherein the first oral care composition and the second oral care composition are applied to the surface of teeth by means of a toothbrush, a swab, a finger cot, a tube applicator, a bleaching tray, or a dental instrument.
